(19) United States
(12) Patent Application Publication
(10) Pub. No.: US 2026/0306751 A1
(43) Pub. Date: Oct. 8, 2026

(54) Sub-assembly of a medicament delivery device with rotatable cap

(71) Applicant: SHL Medical AG, Zug (CH)

(72) Inventor: Meng-Jhen Chiou, Taoyuan City (TW)

(21) Appl. No.: 19/481,931

(22) PCT Filed: May 10, 2024
(86) PCT No.: PCT/EP2024/062911

§ 371 Date: Nov. 6, 2025

(30) Foreign Application Priority Data
May 24, 2023 (EP) 23175011.8

(51) Int. Cl. A61M 5/32; A61M 5/20; A61M 5/315

(52) U.S. Cl. CPC A61M 5/3202; A61M 5/2033; A61M 5/31528

(57) ABSTRACT

The present disclosure provides a sub-assembly of a medicament delivery device, the sub-assembly comprising: a delivery member cover, a rotator, and a cap. The delivery member cover comprises a tubular proximal portion and a distal portion extending from the tubular proximal portion in a direction of a longitudinal axis L. The cap comprises a cap body releasbly covering a proximal end of the tubular proximal portion of the delivery member cover. The cap body comprises a user-accessible surface and a radially directed surface adjacent to a first radially directed surface of the delivery member cover such that when a user of the medicament delivery device rotates the cap via the user-accessible surface around the longitudinal axis, the delivery member cover and the rotator are rotated around the longitudinal axis.

### TECHNICAL FIELD

The present disclosure generally relates to a sub-assembly of a medicament delivery device, and particularly to a sub-assembly of a medicament delivery device comprising a rotator.

### BACKGROUND

Medicament delivery devices such as pen-type manual injectors or auto-injectors are generally known for the self-administration of a medicament by patients without formal medical training. For example, patients suffering from diabetes may require repeated injections of insulin, or patients may require regular injections of other types of medicaments, such as growth hormones.

It is an advantage when the medicament delivery devices for self-administration comprise automatic functions so that even if users are without professional training or are in an emergency, the users can easily and properly use the medicament delivery devices.

Even though many of the devices on the market, as well as the ones described above, have their respective advantages, there is still room for improvement.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device, the sub-assembly comprising: a delivery member cover, a rotator, and a cap. The delivery member cover comprises a tubular proximal portion and a distal portion extending from the tubular proximal portion in a direction of a longitudinal axis; wherein the tubular proximal portion comprises a first radially directed surface facing in a circumferential direction around the longitudinal axis; wherein the distal portion comprises a second radially directed surface facing in the circumferential direction around the longitudinal axis. The rotator comprises a body having a radially directed surface adjacent to the second radially directed surface of the delivery member cover. The cap comprises a cap body releasably covering a proximal end of the tubular proximal portion of the delivery member cover; and wherein the cap body comprises a user-accessible surface and a radially directed surface; wherein the radially directed surface adjacent is to the first radially directed surface of the delivery member cover when the cap is operably attached to the delivery member cover such that when a user of the medicament delivery device rotates the cap via the user-accessible surface around the longitudinal axis, the delivery member cover and the rotator are rotated around the longitudinal axis.

Preferably, according to another embodiment, the body of the rotator comprises a groove/slot extending in the direction of the longitudinal axis.

Preferably, according to another embodiment, the radially directed surface of the rotator is defined by an edge of the groove/slot.

Preferably, according to another embodiment, the distal portion of the delivery member cover comprises a protrusion having the second radially directed surface.

Preferably, according to another embodiment, the protrusion is positioned within the groove/slot.

Preferably, according to another embodiment, a recess/cut-out is arranged in a wall of the tubular proximal portion of the delivery member cover.

Preferably, according to another embodiment, the first radially directed surface is defined by an edge of the recess/cut-out.

Preferably, according to another embodiment, the cap body comprises a protrusion extending into the recess/cut-out of the delivery member cover.

Preferably, according to another embodiment, the protrusion comprises the radially directed surface.

Preferably, according to another embodiment, the sub-assembly comprises a housing extending along the longitudinal axis between a proximal end and a distal end.

Preferably, according to another embodiment, the delivery member cover is positioned at least partially within the housing.

Preferably, according to another embodiment, the tubular proximal portion is telescopic relative to the proximal end of the housing.

Preferably, according to another embodiment, the delivery member cover is rotatable by the cap from a first position to a second position relative to the housing.

Preferably, according to another embodiment, the housing comprises a guiding member; and wherein the delivery member cover comprises a counter guiding member being spaced apart from the guiding member when the delivery member cover is in the first position and being engaged with the guiding member when the delivery member cover is in the second position such that the delivery member cover is rotationally immovable relative to the housing when the delivery member cover is in the second position.

Preferably, according to another embodiment, one of the guiding member and the counter-guiding member is a recess/slot extending in the direction of the longitudinal axis; and the other one of the guiding member and the counter-guiding member is a rib.

Preferably, according to another embodiment, the guiding member is a rib extending from an inner surface of the housing and the counter guiding member is a recess extending on the delivery member cover.

Preferably, according to another embodiment, the sub-assembly comprises a biasing member extending, in the direction of the longitudinal axis, from a surface of the housing to a surface of the delivery member cover.

Preferably, according to another embodiment, the cap is releasably attached to the proximal end of the housing.

Preferably, according to another embodiment, when the cap is attached to the housing, the cap is configured to block a proximal movement of the delivery member cover caused by the biasing member.

Preferably, according to another embodiment, the sub-assembly comprises a plunger rod having a proximally directed surface.

Preferably, according to another embodiment, the rotator comprises a first distally directed surface.

Preferably, according to another embodiment, the rotator is rotatable by the delivery member cover from a first position of the rotator where the proximally directed surface of the plunger rod is engaged with the first distally directed surface of the rotator to a second position of the rotator where the proximally directed surface of the plunger rod is disengaged from the first distally directed surface of the rotator.

Preferably, according to another embodiment, the plunger rod is configured to be connected to an energy source of the medicament delivery device such that the plunger rod is configured to be moved towards the proximal end of the housing by the energy source.

Preferably, according to another embodiment, the rotator comprises a second distally directed surface proximally positioned relative to the first distally directed surface.

Preferably, according to another embodiment, the proximally directed surface of the plunger rod is configured to be moved in to be engaged with the second distally directed surface when the rotator is in the second position of the rotator by the energy source.

Preferably, according to another embodiment, the rotator comprises a proximally directed surface helically extending on a surface of the body of the rotator.

Preferably, according to another embodiment, the distal portion of the delivery member cover comprises a distally directed surface configured to be moved along the proximally directed surface of the rotator when the delivery member cover is in the second position of the delivery member cover such that the rotator is further rotated from the second position of the rotator to a third position of the rotator by a distal movement of the delivery member cover.

Preferably, according to another embodiment, the proximally directed surface of the plunger rod is disengaged from the second distally directed surface of the rotator when the rotator is in the third position.

Preferably, according to another embodiment, the sub-assembly is used in a medicament delivery device.

Preferably, according to another embodiment, the medicament delivery device comprises a medicament container.

Preferably, according to another embodiment, the medicament container is a multiple-chamber medicament container.

Preferably, according to another embodiment, the medicament container is made of glass material or plastic material.

Preferably, according to another embodiment, the medicament container is a syringe, a cartridge or a collapsible bag.

Preferably, according to another embodiment, the medicament container comprises a medicament delivery member attached at a proximal end of the medicament container.

Preferably, according to another embodiment, the medicament delivery device comprises a medicament delivery member attached at the proximal end of the housing.

Preferably, according to another embodiment, the medicament delivery device comprises a medicament delivery member isolated assembly attached at the proximal end of the housing.

Preferably, according to another embodiment, the medicament delivery member isolated assembly comprises a delivery member, a delivery member hub attached to the delivery member, a channel having a fastener engaged with a counter fastener of the delivery member hub, and a delivery member cap covering the delivery member and interacting with the delivery member hub.

Preferably, according to another embodiment, the fastener is a thread, and the counter fastener is a thread follower.

Preferably, according to another embodiment, the fastener is a bayonet track, and the counter fastener is a bayonet follower.

Preferably, according to another embodiment, the delivery member cap is configured to be removed by rotating relative to the housing.

Preferably, according to another embodiment, the delivery member cap is attached to the cap.

Preferably, according to another embodiment, the rotation of the delivery member cap results in the delivery member hub moving towards the medicament container via the interaction with the channel.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the delivery member is an injection needle or a spray nozzle.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device comprising a sub-assembly of the invention;

FIG. 2 schematically shows a perspective view of components of the medicament delivery device of FIG. 1;

FIG. 3 schematically shows a perspective view of a distal portion of a delivery member guard of the sub-assembly of FIG. 1 with a rotator of the sub-assembly of FIG. 1;

FIG. 4 schematically shows a perspective view of a tubular proximal portion of a delivery member guard of the sub-assembly of FIG. 1 with a cap of the sub-assembly of FIG. 1;

FIGS. 5-6 schematically show perspective views of the delivery member and the rotator in two examples respectively;

FIG. 7 schematically shows a perspective view of the rotator of FIG. 3 and a plunger rod of the sub-assembly of FIG. 1;

FIG. 8 schematically shows a perspective view of the distal portion of a delivery member guard of FIG. 3 with a rotator of FIG. 3.

### DETAILED DESCRIPTION

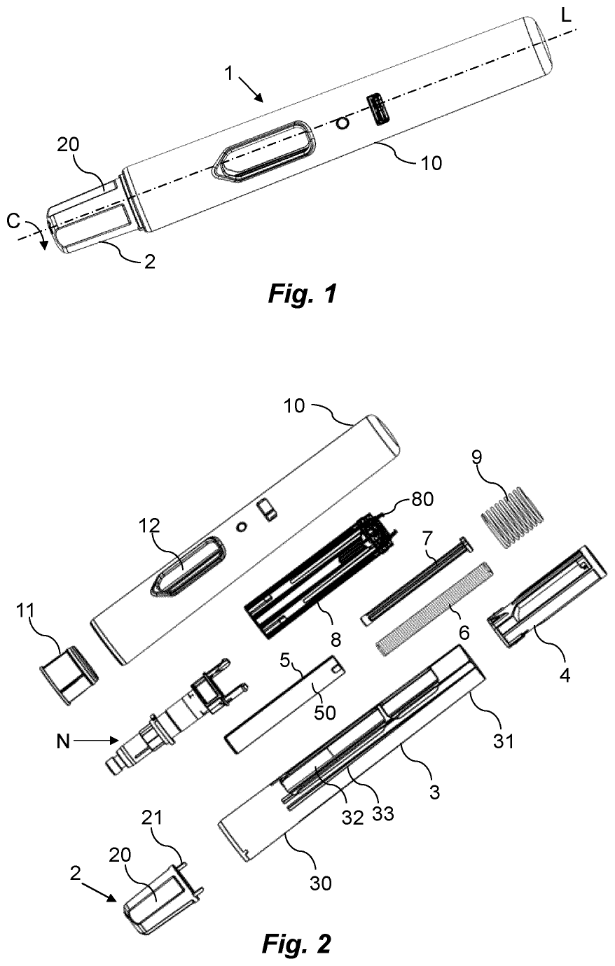

FIGS. 1-8 illustrate a sub-assembly of a medicament delivery device. The sub-assembly comprises a delivery member cover 3, a rotator 4, and a cap 2.

The delivery member cover comprises a tubular proximal portion 30 and a distal portion 31 extending from the tubular proximal portion 30 in a direction of a longitudinal axis L, as shown in FIG. 2. The tubular proximal portion 30 of the delivery member cover 3 is configured to surround a medicament delivery member, e.g., an injection needle or a spray nozzle, of the medicament delivery device that comprises the sub-assembly such that ambient contamination of the medicament delivery member can and/or injury of users of the medicament delivery device can be avoided. In a preferred example, the tubular proximal portion 30 is telescopically arranged relative to a housing 1 of the medicament delivery device. In this example, the housing 1 extends along the longitudinal axis between a proximal end from which the medicament delivery member is configured to protrude during medicament delivery operation and a distal end opposite to the proximal end in the direction of the longitudinal axis L. The tubular proximal portion 30 of the delivery member cover 3 is configured to be telescopically arranged relative to the proximal end of the housing 1. In this example, the tubular proximal portion 30 is movable relative to the housing 1 in the direction of the longitudinal axis L between an extended position where the tubular proximal portion 30 is configured to fully surround the delivery member of the medicament delivery device and a retracted position where the medicament delivery member of the medicament delivery device can at least partially protrude from the tubular proximal portion 30 in the direction of the longitudinal axis L. It should be noted that the tubular proximal portion may be arranged with multiple positions between the extended position and the retracted position dependent on the design.

The distal portion 31 of the delivery member cover 3 is configured to interact with other components of the medicament delivery device dependent on the design. In one example, the distal portion 31 of the delivery member cover 3 is tubular; in this example, the tubular proximal portion 30 of the delivery member cover 3 and the distal portion 31 of the delivery member cover 3 form a sleeve, as shown in FIG. 2. In this example, the delivery member cover comprises a window 32 configured to be lined up with a medicament container arranged within the housing 1. Alternatively, the delivery member cover can be transparent. Alternatively, the distal portion of the delivery member cover does not need to be tubular. For example, the distal portion of the delivery member cover can be one or more straight arms extending from the tubular proximal portion of the delivery member cover.

The tubular proximal portion 30 comprises a first radially directed surface 30a facing in a circumferential direction C around the longitudinal axis L. The distal portion 31 comprises a second radially directed surface 31a facing in the circumferential direction C around the longitudinal axis L. Therefore, when the delivery member cover 3 is rotated by force that applies on the first radially directed surface 30a, delivery member cover 3 can cause another component to rotate via an engagement between the second radially directed surface and the other component.

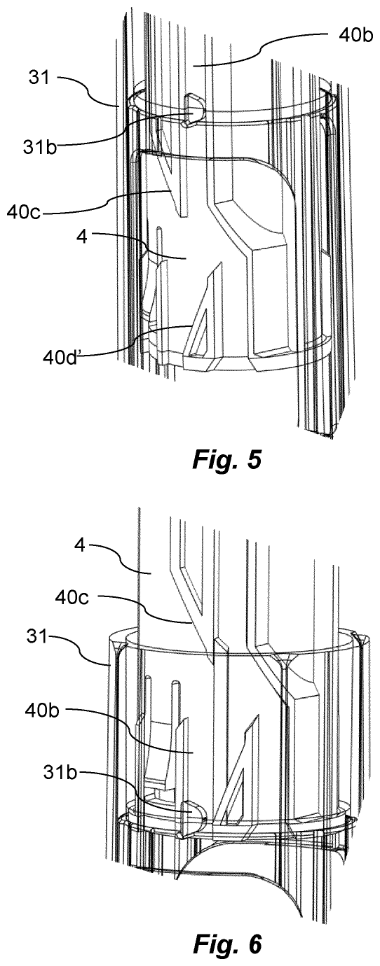

As shown in FIGS. 4-5, the rotator 4 comprises a body 40 having a radially directed surface 40a adjacent to the second radially directed surface 31a of the delivery member cover 3. Furthermore, the cap 2 comprises a cap body 20 releasably covering a proximal end of the tubular proximal portion 30 of the delivery member cover 3, as shown in FIG. 1. The cap body 20 comprises a user-accessible surface 22 and a radially directed surface 21a, as shown in FIG. 4, the radially directed surface 21a is adjacent to the first radially directed surface 30a of the delivery member cover 3 when the cap is operably attached to the delivery member cover 3 as shown in FIG. 1, namely that the cap body is attached to the housing and/or tubular proximal portion of the delivery member cover. As a result, when a user of the medicament delivery device rotates the cap 2 via the user-accessible surface 22 around the longitudinal axis L, the delivery member cover 3 and the rotator 4 are rotated around the longitudinal axis L.

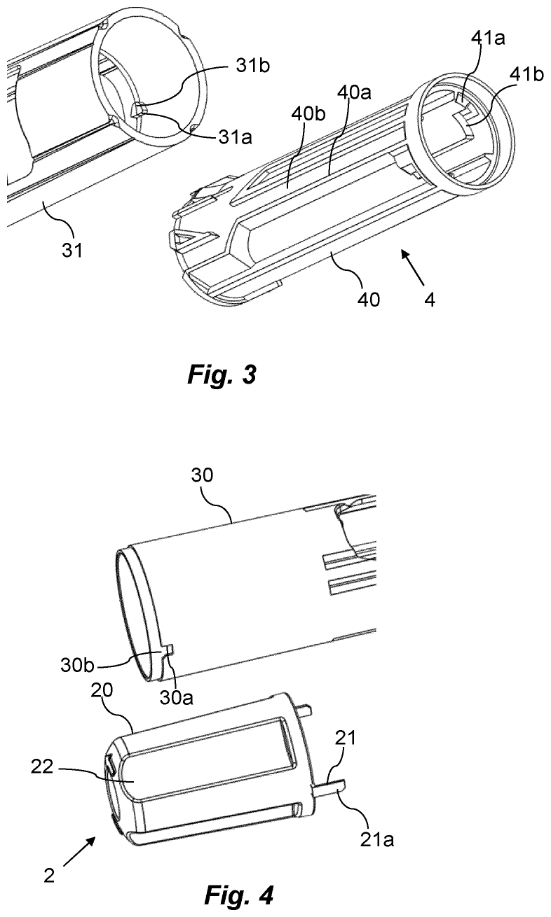

In one example, the body 40 of the rotator 4 comprises a groove/slot 40b extending in the direction of the longitudinal axis L, as shown in FIG. 3 and FIGS. 5-6. In this example, the radially directed surface 40a of the rotator 4 is defined by an edge of the groove/slot 40b. The distal portion 31 of the delivery member cover 3 comprises a protrusion 31b having the second radially directed surface 31a, as shown in FIG. 3. In this example, the protrusion 31b is positioned within the groove/slot 40b.

In another example, a recess/cut-out 30b is arranged in a wall of the tubular proximal portion 30 of the delivery member cover 3. The first radially directed surface 30a is defined by an edge of the recess/cut-out 30b. In this example, the cap body 20 comprises a protrusion 21 extending into the recess/cut-out 30b of the delivery member cover 3. In this example, the protrusion 21 comprises the radially directed surface 21a. Alternatively, the recess/cut-out can be arranged in the cap body; and the tubular proximal portion of the delivery member cover comprises the protrusion positioned within the recess/cut-out.

As mentioned above, when the user manually rotates the cap 2 relative to the housing 1 about the longitudinal axis L, the delivery member cover 3 and the rotator 4 will also be rotated. The rotation of the rotator can be used for different mechanisms. In one example, the sub-assembly comprises a biasing member, e.g., a spring, configured to bias the rotator in the proximal direction relative to the housing. In this example, the rotator is operably engaged with a medicament container of the medicament delivery device. In this example, the rotator is initially positioned on a distally directed surface of the housing such that a proximal movement of the rotator is prevented. When the user manually rotates the cap and thereby the rotator, the rotation of the rotator causes the rotator to disengage from the distally directed surface of the housing; as a result, the biasing member urges the rotator and the medicament container to move in the proximal direction relative to the housing. In this example, the medicament container can be a cartridge. In this example, the medicament delivery device may comprise a delivery member, preferably, a needle, wrapped by a rubber sheath. In a preferred example, the wrapped needle is attached to the proximal end of the housing; thus, the proximal movement of the rotator and the medicament container results in establishing fluid communication between the needle and the medicament container as the biasing member pushes the medicament container, via the rotator, to be penetrated by the needle.

Alternatively, the rotator 4 is axially immovable relative to the housing 1. For example, the rotator 4 is sandwiched by a distally directed surface of the housing, e.g., a ledge protruding from the inner wall of the housing, and a proximally directed surface of the housing, e.g., an inner rear end wall of the housing. Alternatively, the rotator 4 is sandwiched by a distal end of the container carrier 8 and a proximally directed surface of the housing, e.g., an inner rear end wall of the housing.

In one example where the rotator is axially immovable relative to the housing, the rotation of the rotator 4 caused by the delivery member cover 3 can be used to release other components, e.g., a plunge rod, that is engaged with the rotator 4. This example will be further explained later.

In one example, the cap is releasably attached to the proximal end of the housing. In a preferred example, the rotation of the cap can be designed as an initial step of the use of the medicament delivery device. For example, the cap is designed to the removed from the housing via a rotation, e.g., the cap can be attached to the housing via a thread or a bayonet engagement. Thus, in one example where the rotation of the rotator is initiated by the manual rotation of the cap, when the user removes the cap, the needle can be automatically mounted to the medicament container.

In a preferred example, the sub-assembly comprises the housing 1 of the medicament delivery device. The housing 1 comprises a housing body 10 and a front body 11. In one example, the housing body 10 and the front body 11 are two components configured to be attached, as shown in FIG. 2. In one example, the sub-assembly comprises a medicament container carrier 8 for accommodating a medicament container. In one example, the medicament container carrier 8 is attached to the housing 1. Alternatively, the medicament container is an integral part of the housing.

Alternatively, the front body is an integral part of the housing body. In a preferred example, a window 12 is arranged in the wall of the housing body 10 such that the user of the medicament delivery device can observe a medicament container within the housing 1. In a preferred example, the delivery member cover 3 is rotatable by the cap 2 from a first position to a second position relative to the housing 1. In this example, the housing 1 comprises a guiding member and the delivery member cover 3 comprises a counter-guiding member 33 is spaced apart from the guiding member when the delivery member cover 3 is in the first position. The counter-guiding member 33 of the delivery member cover 3 engaged with the guiding member when the delivery member cover 3 is in the second position such that the delivery member cover 3 is rotationally immovable relative to the housing 1 when the delivery member cover 3 is in the second position. In one example, the guiding member is a rib extending in the direction of the longitudinal axis L. In this example, the guiding member extends from an inner surface of the housing body 10. In this example, the counter-guiding member 33 is a recess extending in the direction of the longitudinal axis on the delivery member cover 3, as shown in FIG. 2. Alternatively, the guiding member can be the recess and the counter-guiding member can be the rib.

In this example, once the delivery member cover 3 is manually rotated by the user via the cap 2 into the second position, the delivery member cover 3 cannot be rotated relative to the housing 1 as the counter guiding member 33 is engaged with the guiding member. As a result, the delivery member cover 3 can only be moved linearly relative to the housing 1. Therefore, further linear movement of the delivery member cover 3 can be used to rotate the rotator 4 as explained below.

In this example, the rotator is axially immovable relative to the housing after the initial rotator is initiated by the manual rotation of the cap. For example, the rotator can be sandwiched between a proximal directed surface of the housing and a medicament container of the medicament delivery device. In this example, the rotator may be always axially immovable relative to the housing once the medicament delivery device is assembled. Alternatively, in one example where the initial rotation of the rotator results in an axial movement of the rotator as mentioned above, the axial movement of the rotator may result in that the rotator moves past a wedge structure extending from the inner surface of the housing. Thus, the rotator is blocked from moving in the distal direction relative to the housing after the rotator moves past the wedge structure. In this example, further proximal movement of the rotator is blocked by the medicament container that is engaged with the housing after the initial axial movement. Thus, the rotator is axially immovable relative to the housing after the initial rotation and the initial axial movement

In one example where the sub-assembly comprises the housing 1 of the medicament delivery device, as mentioned above, the tubular proximal portion 30 of the delivery member cover 3 is telescopically relative to the proximal end of the housing 1. In this example, a biasing member 9 is extending, in the direction of the longitudinal axis, from a surface of the housing 1 to a surface of the delivery member cover 3. The basing member can be a spring, a compressions spring, a tension spring, a torsion spring, or a flexible arm.

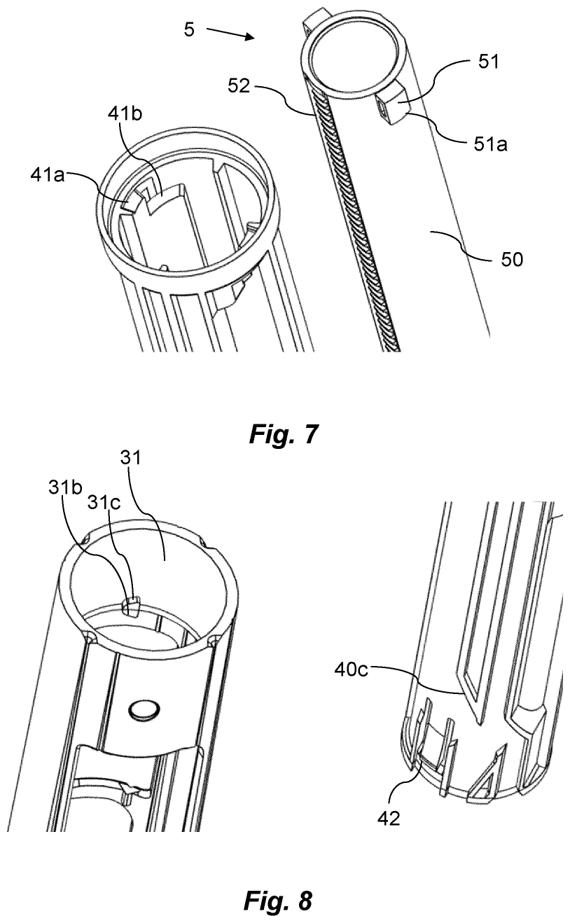

In one example, the sub-assembly comprises a plunger rod 5 having a proximally directed surface 51a, as shown in FIG. 7. The plunger rod 5 is configured to connect to an energy source of the medicament delivery device such that the plunger rod 5 is configured to be moved towards the proximal end of the housing 1 by the power source. In one example, the plunger rod 5 comprises a tubular body 50 configured to receive an energy source 6 within the tubular body 50. In one example, the energy source 6 is a spring positioned within the tubular body 50 of the plunger rod 5. In a preferred example, when the energy source 6 is a spring, the sub-assembly comprises a guide rod 7 position within the spring 6 to avoid the spring 6 from buckling. Alternatively, the energy source is propellant gas configured to flow into the tubular body of the plunger rod from a gas canister during the use of the medicament delivery device. Alternatively, the energy source is configured to position outside of the plunger rod; in this example, the plunger rod does not need a tubular body. At least a part of the plunger rod 5 is configured to move into the medicament container of the medicament delivery device to expel the medicament contained within the medicament container. In one example, the plunger rod 5 comprises a radial protrusion 51 extending away from the longitudinal axis L, as shown in FIG. 7. In this example, the radial protrusion 51 comprises the proximally directed surface 51a. Furthermore, the plunger rod may comprise a feedback element 52, e.g., ratchet, one or more clicking teeth/arms, configured to interact with another component of the medicament delivery device to indicate the user of the medicament delivery device status of the medicament delivery device. For example, the feedback element 52 is a set of ratchets extending in the direction of the longitudinal axis L and configured to interact with an arm extending from the housing during the medicament delivery operation. Alternatively, instead of extending from the housing, the arm 80 can extend from the container carrier 8.

In one example where the plunger rod 5 comprises the proximally directed surface 51a, the rotator 4 comprises a first distally directed surface 41a. In this example, the rotator 4 is rotatable by the delivery member cover 3 from a first position where the proximally directed surface 51a of the plunger rod 5 is engaged with the first distally directed surface 41a of the rotator 4 to a second position where the proximally directed surface 51a of the plunger rod 5 is disengaged from the first distally directed surface 41a of the rotator 4. As mentioned above, when the user manually rotates the cap 2 relative to the housing 1, the delivery member cover 3 is rotated. As a result, the rotation of the delivery member cover 3 rotates the rotator 4 via the engagement between the second radially directed surface 31a and the radially directed surface 40a of the rotator 4. The rotation of the rotator 4 enables the proximal movement of the plunger rod 5; thus, the plunger rod 5 can be moved in the proximal direction relative to the housing 1 by the energy source. In one example where the cap 2 is configured to be removed by being rotated relative to the housing 1 as mentioned above and the energy source is a spring when the user removes the cap 2 from the housing 1, the plunger rod 5 is moved in the proximal direction, preferably, into the medicament container; as a result, a priming function (contained air expelling) and/or a mixing function (reconstituting at least two medicaments within the medicament container) can be automatically carried out.

In another preferred example, the rotator 4 comprises a second distally directed surface 41b proximally positioned relative to the first distally directed surface 41a, as shown in FIG. 3 and FIG. 7. The proximally directed surface 51a of the plunger rod 5 is configured to be moved in to be engaged with the second distally directed surface 41b when the rotator 4 is in the second position by the energy source. In one example where the delivery member cover 3 is rotationally immovable relative to the housing 1 after the delivery member cover 3 is rotated into the second position of the delivery member cover 3, the further movement of the delivery member cover 3 is configured to release the plunger rod 5 from the second distally directed surface 41b of the rotator 4. In a preferred example, the distal linear movement of the delivery member cover 3 is configured to release the plunger rod from the second distally directed surface 41b of the rotator 4. In one example, the rotator 4 comprises a proximally directed surface 40c helically extending on a surface of the body 40 of the rotator 4, as shown in FIGS. 5-6 and FIG. 8. In this example, the distal portion 31 of the delivery member cover 3 comprises a distally directed surface 31c, as shown in FIG. 8, configured to be moved along the proximally directed surface 41c of the rotator 4 when the delivery member cover 3 is in the second position of the delivery member cover 3 such that the rotator 4 is further rotated from the second position of the rotator 4 to a third position of the rotator 3 by a distal movement of the delivery member cover 3. As a result, the proximally directed surface 51a of the plunger rod 5 is disengaged from the second distally directed surface 41b of the rotator 4; thus, the plunger rod 5 can be moved further in the proximal direction relative to the housing 1 by the energy source 6. In a preferred example, the further proximal movement of the plunger rod 5 is configured to expel the medicament contained within the medicament container.

In a preferred example, the protrusion 31b of the delivery member cover 3 has the second radially directed surface 31a and also has the distally directed surface 31c. In a preferred example, the rotator 4 comprises a ledge extending from an inner surface of the body 40 of the rotator towards the longitudinal axis L, the ledge comprises the first distally directed surface 41a of the rotator 4 and/or the second distally directed surface 41b of the rotator 4.

In a preferred example, the rotator 4 comprises a locking arm 42 radially flexible in the direction transverse to the longitudinal axis L. The locking arm 42 is circumferentially offset from the proximal surface 40c of the rotator 4. In a preferred example, the distally directed surface 31c of the delivery member cover 3 is configured to move along the helical, proximal surface 40c of the rotator 4 to cause the rotator 4 to further rotate from the second position of the rotator 4 to the third position of the rotator 4. The further rotation of the rotator can be used to enable the plunger rod 5 to be moved further in the proximal direction as mentioned above; and/or to align the locking arm 42 with the protrusion 31b of the delivery member cover 3 in the direction of the longitudinal axis L. As a result, when the delivery member cover 3 is moved from the retracted position towards the extended position by the biasing member 9, the protrusion 31b moves past the locking arm 42 of the rotator 4. As a result, any further distal movement of the delivery member cover 3 is blocked by the distally directed surface 31c of the delivery member cover 3 and a proximally directed surface of the locking arm 42 of the rotator 4.

Furthermore, in another example, the delivery member cover 3 can be pre-tensioned before the cap 2 is removed from the housing 1. In this example, the cap 2 is configured to be removed by being rotated relative to the housing 1. Thus, the proximal movement of the delivery member cover 3 is blocked by the cap 2. In this example, as shown in FIG. 5, the rotator 4 comprises a distally directed helical surface 40d′. In this example, when the cap 2 is removed from the housing 1, the delivery member cover 3 is moved in the proximal direction relative to the housing 1 by the biasing member 9 towards the extended position, the protrusion 31b of the delivery member cover 3 is moved along the distally directed helical surface 40d′ of the rotator 4, thus, causes the rotator 4 to rotate. In this example, the rotator 4 is configured to be rotated at least three times in the following order: (1) during the rotation of the cap; (2) during delivery member cover being moved towards the extended position when the cap is removed; and (3) during the delivery member cover being moved towards the retracted position. In this example, multiple automatic functions can be provided. For example, the first rotation of the rotator during the rotation of the cap can result in establishing fluid communication between the medicament delivery member and the medicament container; the second rotation of the rotator during delivery member cover is moved towards the extended position when the cap is removed can result in releasing the plunger rod from the first distally directed surface of the rotator and thus the priming function is carried out; and the third rotation of the rotator can result in releasing the plunger rod from the second distally directed surface of the rotator and thus expelling the medicament to the user. In one example, the sub-assembly can be standardized. In this example, the rotator can be rotated three times in all cases regardless the medicament delivery devices need all three functions or only need one or two. For example, the first rotation (1) of the rotator may result in release the plunger rod from the first distally directed surface of the rotator to carry out the priming and/or mixing function; the second rotation (2) of the rotator may not cause any other component to move; and the third rotation (3) of the rotator may result in releasing the plunger rod for expelling the medicament. In this example, the second rotation (2) of the rotator is a redundant action for those medicament delivery devices that only need the priming and/or mixing function and expelling function.

Alternatively, for those medicament delivery devices that only need the rotator to rotate twice, the rotator 4 doesn't need the distally directed helical surface 40d′ of the rotator 4, as shown in FIG. 6. In this example, the delivery member cover 3 is in the extended position when the cap 2 is attached to the housing 1. For example, the medicament container is a syringe (the medicament delivery member is always in fluid communication with the contained medicament), or the medicament delivery device comprises a medicament delivery set N with a fluid communication establishing mechanism, e.g., a needle isolated assembly as disclosed in U.S. Pat. No. 8,708,970B2; in these two examples, the rotator 4 can only be rotated two times, one for priming/mixing; and the other one for medicament expelling.

Furthermore, the housing of the medicament delivery device, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of medicaments that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives.

Exemplary medicaments that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example, pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary medicaments that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary medicaments that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary medicaments that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary medicaments that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy medicaments include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A sub-assembly of a medicament delivery device, the sub-assembly comprising:
a delivery member cover;
a rotator; and
a cap,
wherein the delivery member cover comprises a tubular proximal portion and a distal portion extending from the tubular proximal portion in a direction of a longitudinal axis,
wherein the tubular proximal portion comprises a first radially directed surface facing in a circumferential direction around the longitudinal axis,
wherein the distal portion comprises a second radially directed surface facing in the circumferential direction around the longitudinal axis;
wherein the rotator comprises a body having a radially directed surface adjacent to the second radially directed surface of the delivery member cover
wherein the cap comprises a cap body releasably covering a proximal end of the tubular proximal portion of the delivery member cover,
wherein the cap body comprises a user-accessible surface and a radially directed surface, and
wherein the radially directed surface adjacent is to the first radially directed surface of the delivery member cover when the cap is operably attached to the delivery member cover such that when a user of the medicament delivery device rotates the cap via the user-accessible surface around the longitudinal axis, the delivery member cover and the rotator are rotated around the longitudinal axis.

17. The sub-assembly according to claim 16, wherein the body of the rotator comprises a groove/slot extending in the direction of the longitudinal axis, wherein the radially directed surface of the rotator is defined by an edge of the groove/slot, wherein the distal portion of the delivery member cover comprises a protrusion having the second radially directed surface and wherein the protrusion is positioned within the groove/slot.

18. The sub-assembly according to claim 16, wherein a recess/cut-out is arranged in a wall of the tubular proximal portion of the delivery member cover, wherein the first radially directed surface is defined by an edge of the recess/cut-out, wherein the cap body comprises a protrusion extending into the recess/cut-out of the delivery member cover, and wherein the protrusion comprises the radially directed surface.

19. The sub-assembly according to claim 16, comprising a housing extending along the longitudinal axis between a proximal end and a distal end, wherein the delivery member cover is positioned at least partially within the housing, and wherein the tubular proximal portion is telescopic relative to the proximal end of the housing.

20. The sub-assembly according to claim 19, wherein the delivery member cover is rotatable by the cap from a first position to a second position relative to the housing; wherein the housing comprises a guiding member, and wherein the delivery member cover comprises a counter guiding member being spaced apart from the guiding member when the delivery member cover is in the first position and being engaged with the guiding member when the delivery member cover is in the second position such that the delivery member cover is rotationally immovable relative to the housing when the delivery member cover is in the second position.

21. The sub-assembly according to claim 20, wherein the guiding member is a recess/slot extending in the direction of the longitudinal axis, and wherein the counter-guiding member is a rib extending away from the longitudinal axis.

22. The sub-assembly according to claim 19, comprising a biasing member is extending, in the direction of the longitudinal axis, from a surface of the housing to a surface of the delivery member cover.

23. The sub-assembly according to claim 19, wherein the cap is releasably attached to the proximal end of the housing.

24. The sub-assembly according to claim 22, when the cap is attached to the housing, the cap is configured to block a proximal movement of the delivery member cover caused by the biasing member.

25. The sub-assembly according to claim 16, comprising a plunger rod having a proximally directed surface; wherein the rotator comprises a first distally directed surface, and wherein the rotator is rotatable by the delivery member cover from a first position of the rotator where the proximally directed surface of the plunger rod is engaged with the first distally directed surface of the rotator to a second position of the rotator where the proximally directed surface of the plunger rod is disengaged from the first distally directed surface of the rotator.

26. The sub-assembly according to claim 25, wherein the plunger rod is configured to be connected to an energy source of the medicament delivery device such that the plunger rod is configured to be moved towards the proximal end of the housing by the energy source.

27. The sub-assembly according to claim 26, wherein the rotator comprises a second distally directed surface proximally positioned relative to the first distally directed surface, and wherein the proximally directed surface of the plunger rod configured to be moved in to be engaged with the second distally directed surface when the rotator is in the second position of the rotator by the energy source.

28. The sub-assembly according to claim 27, wherein the rotator comprises a proximally directed surface helically extending on a surface of the body of the rotator, and wherein the distal portion of the delivery member cover comprises a distally directed surface configured to be moved along the proximally directed surface of the rotator when the delivery member cover is in the second position of the delivery member cover such that the rotator is further rotated from the second position of the rotator to a third position of the rotator by a distal movement of the delivery member cover.

29. The sub-assembly according to claim 28, wherein the proximally directed surface of the plunger rod is disengaged from the second distally directed surface of the rotator when the rotator is in the third position.

30. A medicament delivery device comprising the sub-assembly according to claim 16.
